(19) United States
(12) Patent Application Publication
(10) Pub. No.: US 2026/0051387 A1
(43) Pub. Date: Feb. 19, 2026

(54) METABOLOMIC ANALYSIS OF PERITONEAL DIALYSIS EFFLUENT

(71) Applicant: Fresenius Medical Care Holdings, Inc., Waltham, MA (US)

(72) Inventors: Nadja Grobe, New York, NY (US); Peter Kotanko, New York, NY (US); Xiaoling Wang, New York, NY (US)

(73) Assignee: Fresenius Medical Care Holdings, Inc., Waltham, MA (US)

(21) Appl. No.: 19/300,947

(22) Filed: Aug. 15, 2025

Related U.S. Application Data
(60) Provisional application No. 63/683,341, filed on Aug. 15, 2024.

(51) Int. Cl. G16H 20/40; A61M 1/28; G16B 40/00; G16C 20/70; G16H 10/60

(52) U.S. Cl. CPC G16H 20/40; A61M 1/28; G16B 40/00; G16C 20/70; G16H 10/60

(57) ABSTRACT

Methods, apparatuses, and systems for determining a peritoneal transport status classification of a patient based on mass analyzing low volumes of peritoneal dialysis (PD) effluent evaluated using PD effluent fingerprints of known transport statuses to determine a peritoneal transport status classification of the patient are described. In one example, a method includes obtaining a volume of PD effluent of the dialysis patient, generating patient information via mass analysis of the volume of PD effluent, and determining patient profile information based on evaluating the patient information with a profile library, the patient profile information comprising a peritoneal transport status classification. The PD effluent fingerprints may include information of at least one biomarker including at least one of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, and 4-hydroxybenzoic acid.

### CROSS REFERENCE TO RELATED APPLICATIONS

This application claims the benefit of U.S. Provisional Patent Application No. 63/683,341, filed Aug. 15, 2024, and entitled “Metabolomic Analysis of Peritoneal Dialysis Effluent,” the entirety of which is incorporated by reference herein.

### FIELD OF THE DISCLOSURE

The disclosure generally relates to determining physical characteristics of dialysis patients, and more particularly to processes for determining patient dialysis profile information indicative of the health of the patient and/or the success of peritoneal dialysis (PD) for the patient.

### BACKGROUND

Patient treatment success in PD is dependent on the functional and morphological integrity of the peritoneal membrane. In addition to functional failure of the peritoneum, long-term PD may lead to anatomical changes in the peritoneal tissues such as neoangiogenesis, vasculopathy and fibrosis, sometimes causing peritoneal sclerosis. Accordingly, various patient characteristics are typically monitored during the course of PD treatment, including peritoneal transport status (i.e., transport across the peritoneal membrane for various solutes). However, conventional methods for determining peritoneal transport status (and/or other patient characteristics) are labor-intensive, time-consuming, and require extra patient clinic visits outside of regular PD treatment. Accordingly, PD patients and healthcare providers would benefit from processes capable of efficiently and effectively determining patient characteristics that may affect PD treatment without the drawbacks of conventional methods.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure relates generally to methods, apparatuses, and systems for determining a peritoncal transport status of a patient based on mass analyzing low volumes of PD effluent to generate patient information that may be evaluated using PD effluent fingerprints to determine peritoneal transport characteristics of the patient. Other embodiments are described.

In some embodiments, a method disclosed herein includes obtaining a volume of peritoncal dialysis (PD) effluent of a dialysis patient; generating patient information via mass analysis of the volume of PD effluent, the patient information comprising at least one transport status biomarker; determining patient profile information of the dialysis patient based on evaluating the patient information with a profile library of transport status biomarker information of a population of patients with known transport statuses, the patient profile information comprising a peritoncal transport status classification, wherein the transport status biomarker information comprises information for at least one biomarker, the at least one biomarker including at least one of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, or 4-hydroxybenzoic acid; and performing a dialysis treatment on the dialysis patient based on the peritoneal transport status classification of the patient profile information.

In some embodiments of the method, the peritoneal transport status classification is selected from a set of classifications indicating a high, high-average, low-average, or low transporter status based on solute transport characteristics.

In some embodiments of the method, the transport status biomarker information comprises at least one characteristic of the at least one biomarker, the at least one characteristic comprising at least one of presence of the at least one biomarker, a concentration of the at least one biomarker, or regulation of the at least one biomarker.

In some embodiments of the method, the peritoneal transport status classification is indicated using a numerical identifier or symbol. In some embodiments, the method further comprises determining a dialysis prescription based on the peritoneal transport status classification.

In some embodiments, the method further comprises determining a type of PD based on the peritoneal transport status classification; and performing the dialysis treatment on the dialysis patient according to the determined type of PD. In some embodiments, the method further comprises determining a PD solution based on the peritoneal transport status classification; and performing the dialysis treatment on the dialysis patient according to the determined PD solution.

In another embodiment, an apparatus, such as a computer apparatus for determining a peritoneal transport status of a patient. In such an embodiment, the apparatus includes a processing circuit; and a memory coupled to the processing circuit, the memory comprising instructions that, when executed by the processing circuit, cause the processing circuit to perform various operations. In some embodiments, the processing circuit is caused to access a computational model trained using training data comprising biomarker information of peritoneal dialysis (PD) patients with known transport statuses to generate output comprising a transport status determination of a patient indicating the PD functionality of the peritoneal membrane of the patient based on input comprising biomarker information of the patient, wherein the biomarker information includes information on at least one of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, or 4-hydroxybenzoic acid. In some embodiments, the processing circuit is caused to receive biomarker information of a patient from PD effluent of the patient following a PD treatment. In some embodiments, the processing circuit is caused to determine the peritoneal transport status classification for the patient via providing the biomarker information to the computational model. In some embodiments, the processing circuit is caused to send a control signal to a dialysis machine to perform a dialysis treatment on the dialysis patient based on the peritoneal transport status classification of the patient profile information.

In some embodiments, the peritoneal transport status classification is selected from a set of classifications indicating a high, high-average, low-average, or low transporter status based on solute transport characteristics.

In some embodiments, the training data comprises at least one characteristic of the at least one biomarker, the at least one characteristic comprising at least one of presence of the at least one biomarker, a concentration of the at least one biomarker, or regulation of the at least one biomarker.

In some embodiments, the computational model includes one or more of: a machine learning model; an artificial intelligence model; a neural network; or a convolutional neural network.

In some embodiments, the peritoneal transport status classification is indicated using a numerical identifier or symbol. In some embodiments, the biomarker information includes metabolites that demonstrate at least one different characteristic between peritoneal transport status classifications. In some embodiments, the training data includes data resulting from a peritoncal equilibration test (PET) of PD patients.

In another embodiment, a PD machine comprising the any of the apparatuses described above is provided. In this embodiment, the PD machine is configured to process the control signal and effectuate the dialysis treatment based on the peritoneal transport status classification.

In another embodiment, a non-transitory computer-readable storage medium having executable instructions stored thereon is disclosed. In such an embodiment, when the instructions are executed by the processing circuit, it causes the processing circuit to perform various operations. In some embodiments, the processing circuit is caused to access a computational model trained using training data comprising biomarker information of peritoneal dialysis (PD) patients with known transport statuses to generate output comprising a transport status determination of a patient indicating the PD functionality of the peritoneal membrane of the patient based on input comprising biomarker information of the patient, wherein the biomarker information includes information on at least one of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, or 4-hydroxybenzoic acid. In some embodiments, the processing circuit is caused to receive biomarker information of a patient from PD effluent of the patient following a PD treatment. In some embodiments, the processing circuit is caused to determine the peritoneal transport status classification for the patient via providing the biomarker information to the computational model. In some embodiments, the processing circuit is caused to send a control signal to a dialysis machine to perform a dialysis treatment on the dialysis patient based on the peritoneal transport status classification of the patient profile information.

In this embodiment, the peritoneal transport status classification is selected from a set of classifications indicating a high, high-average, low-average, or low transporter status based on solute transport characteristics. In this embodiment, the training data comprises at least one characteristic of the at least one biomarker, the at least one characteristic comprising at least one of presence of the at least one biomarker, a concentration of the at least one biomarker, or regulation of the at least one biomarker.

In this embodiment, the computational model includes one or more of: a machine learning model; an artificial intelligence model; a neural network; or a convolutional neural network. In this embodiment, the peritoneal transport status classification is indicated using a numerical identifier or symbol.

In another embodiment, a method of determining a transport status of a dialysis patient may include obtaining a volume of PD effluent of the dialysis patient, generating patient information via mass analysis of the volume of PD effluent, and determining patient profile information based on evaluating the patient information with a profile library, the patient profile information comprising a peritoneal transport status classification.

In another embodiment, a method of performing dialysis for a dialysis patient may include performing a PD process on the patient based on a peritoneal transport status, the peritoneal transport status determined via obtaining a volume of PD effluent of the dialysis patient, generating patient information via mass analysis of the volume of PD effluent, and determining patient profile information based on evaluating the patient information with a profile library, the patient profile information comprising a peritoneal transport status classification.

In another embodiment, an apparatus may include at least one memory and logic coupled to the at least one memory, the logic to receive patient information generated via mass analysis of a volume of PD effluent of a patient, and determine patient profile information based on evaluating the patient information with a profile library, the patient profile information comprising a peritoneal transport status classification

In some embodiments, the mass analysis comprising one of liquid chromatography-mass spectrometry (LC-MS) or mass spectrometry (MS). In various embodiments, the volume obtained during routine dialysis of a patient. In various embodiments, the volume comprising less than or equal to about 1 milliliter (ml). In some embodiments, the peritoneal transport status classification comprising classifications of high, high-average, low-average, or low transporters based on solute transport characteristics. In various embodiments, a dialysis prescription may be determined based on the peritoneal transport status classification. In some embodiments, a dialysis treatment may be performed on the patient based on the peritoneal transport status classification.

In one example, a method of determining a transport status of a dialysis patient includes: obtaining a volume of PD effluent of the dialysis patient; generating patient information via mass analysis of the volume of PD effluent, the patient information comprising at least one transport status biomarker; and determining patient profile information based on evaluating the patient information with a profile library of transport status biomarker information of a population of patients with known transport statuses, the patient profile information comprising a peritoneal transport status classification.

In some embodiments of the method, the transport status biomarker information includes information for at least one biomarker, the at least one biomarker including at least one of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, and 4-hydroxybenzoic acid.

In various embodiments of the method, the transport status biomarker information includes at least one characteristic of the at least one biomarker, the at least one characteristic comprising at least one of presence of the at least one biomarker, a concentration of the at least one biomarker, or regulation of the at least one biomarker.

In some embodiments of the method, the peritoneal transport status classification includes classifications of high, high-average, low-average, or low transporters based on solute transport characteristics.

In various embodiments of the method, the method further includes determining a dialysis prescription based on the peritoneal transport status classification.

In some embodiments of the method, the method further includes determining a type of PD dialysis based on the peritoneal transport status classification.

In exemplary embodiments of the method, the method further includes determining a PD dialysis solution based on the peritoneal transport status classification.

In one example, an apparatus includes at least one processor, and a memory coupled to the at least one processor. The memory includes instructions that, when executed by the at least one processor, cause the at least one processor to access a computational model trained using training data comprising biomarker information of PD patients with known transport statuses to generate output comprising a transport status determination of a patient indicating the PD functionality of the peritoneal membrane of the patient based on input comprising biomarker information of the patient, receive biomarker information of a patient from PD effluent of the patient following a PD treatment, and determine the transport status determination for the patient via providing the biomarker information to the computational model.

In some embodiments of the apparatus, the training data includes information for at least one biomarker, the at least one biomarker including at least one of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, and 4-hydroxybenzoic acid.

In some embodiments of the apparatus, the training data includes at least one characteristic of the at least one biomarker, the at least one characteristic comprising at least one of presence of the at least one biomarker, a concentration of the at least one biomarker, or regulation of the at least one biomarker.

### BRIEF DESCRIPTION OF THE DRAWINGS

By way of example, specific embodiments of the disclosed machine will now be described, with reference to the accompanying drawings, in which:

FIG. 1 illustrates a first exemplary operating environment according to some embodiments.

FIG. 2 illustrates a PD profile process according to some embodiments.

FIG. 3 illustrates PD profiles according to some embodiments.

FIG. 4A illustrates PD profiles according to some embodiments.

FIG. 4B depicts standardized equilibration curves for creatinine and glucose.

FIG. 5A illustrates targeted and untargeted approaches to a PD profile process according to some embodiments.

FIG. 5B illustrates exemplary mass analysis results of a PD profile process according to some embodiments.

FIG. 6 depicts non-limiting example transport status biomarkers according to some embodiments.

FIG. 7 depicts a workflow for identifying metabolite biomarkers according to some embodiments.

FIG. 8A illustrates an example of a processing flow according to some embodiments.

FIG. 8B illustrates an example of a processing flow according to some embodiments.

FIGS. 9A and 9B illustrate an exemplary PD system according to some embodiments.

FIG. 10 illustrates an embodiment of a computing architecture in accordance with the present disclosure.

FIG. 11 is a flow chart illustrating various operations of a method in accordance with the present disclosure.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete, and willfully convey the scope of the subject matter to those skilled in the art. In the drawings, like numbers refer to like elements throughout.

The present disclosure is directed to systems and methods for PD treatment of patients. In some embodiments, the peritoneal membrane transport profile (or “status”) of a patient may be determined to provide a targeted, personalized PD treatment, treatment recommendation, dialysis prescription, and/or the like for a specific patient based on the patient biomarkers detected or measured in patient fluid. In various embodiments, the patient fluid may be one or more of PD effluent or patient blood. In various embodiments, the biomarker profile of the patient may be used to determine a peritoneal membrane transport status of the patient by evaluating the biomarker profile against a library of biomarker profiles of patients with known peritoneal membrane transport statuses.

In some embodiments, the biomarkers may include metabolomic information. Non-limiting example biomarkers include one or more of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, and 4-hydroxybenzoic acid. The identified biomarkers were discovered using untargeted metabolite profiling, a methodology that does not rely on prior hypotheses and eliminates selection bias. This approach, enabled by access to comprehensive mass spectral libraries, has, for the first time, established that these known molecules are associated with peritoneal dialysis (PD) transporter type. These identified biomarkers represent surprising, new, unexpected, or hard to identify results in developing treatment strategies with respect to patient peritoneal membrane and transport status identification. Other biomarkers have been indicated as being potential biomarkers for indicating peritoncal transport status and/or membrane function.

Patient treatment success in PD is dependent on the functional and morphological integrity of the peritoneal membrane. Traditionally, a new PD patient starts out with a generic PD prescription, for instance, with respect to the number of dialysis solution dwell cycles, dwell duration, dialysis solution characteristics (e.g., glucose strength), and/or the like. Following a testing period, for example, a few months the patient undergoes a peritoneal membrane function assessment. Conventional assessment tests include a standard Peritoneal Equilibrium Test (PET) to determine certain patient PD characteristics, such as peritoneum health or transport characteristics. Non-limiting examples of transport characteristics include a transport rate, such as a high, low/high average, or low peritoneal transport rate. Based on the result of the PET, the patient's PD prescription and technique (e.g., the use of continuous ambulatory peritoneal (CAPD) versus ambulatory PD (APD)) may be adjusted.

A patient's response to their PD treatments may likely change over time because the peritoneum functionality may deteriorate (e.g., based on morphological alteration of the peritoneum when it is used as a dialysis membrane), causing the peritoneum to operate less efficiently as the patient continues their PD treatment.

In addition to functional failure of the peritoneum, long-term PD may lead to anatomical changes in the peritoneal tissues such as neoangiogenesis, vasculopathy and fibrosis, sometimes causing peritoneal sclerosis. Membrane characteristics alter especially after sustained use of non-physiological dialysis fluids. Accordingly, patient characteristics may be monitored over the duration of a PD patient treatment regimen to ensure, among other things, the health of patient peritoneal anatomy and/or the effectiveness of PD treatment. Non-limiting patient characteristics may include peritoneal transport status, dialysis adequacy, membrane characteristics, unexplained clinical changes, ultrafiltration failure, and/or the like. In some embodiments, a treatment recommendation, dialysis prescription, and/or dialysis treatment may be administered based on the patient characteristic determinations according to some embodiments.

The primary monitored characteristic may include peritoneal transport status for PD patients. In general, peritoneal transport status is a classification of membrane function by measuring the rate at which solutes equilibrate between the dialysate and body plasma. For example, the dialysate-to-plasma (D/P) ratio may operate to measure the combined effect of diffusion and ultrafiltration during DP. A low solute D/P means that transport across the peritoneal membrane for a given solute occurs slowly, and equilibrium between the dialysate and plasma is reached gradually. In contrast, a high solute D/P means that transport of a solute across the membrane occurs quickly, and equilibrium is reached sooner. D/P ratios are typically assessed for various solutes including urea, creatinine, and sodium. While these compounds are water-soluble, low-molecular-weight markers, they fail to reflect the transport behavior of molecules with more diverse physicochemical characteristics.

Peritoneal transport parameters provide the basis on which to create and revise a PD prescription that is tailored and optimized for each individual patient. The target PD prescription maintains optimal homeostasis including, for example, removal of sufficient toxins, wastes, salts, and fluid from the patient. Therefore, updating a patient's PD prescriptions over time in response to peritoneum functionality changes to provide more optimized treatment for the patient is helpful for PD success and overall patient health.

Conventional tests for monitoring peritoneal transport status are generally time consuming, difficult for patients, and lack analysis of the full array of elements (for instance, metabolites) that may be used to form a complete assessment. For example, the PET was developed over 30 years ago to assess peritoneal transport status in patients undergoing PD. The standard PET requires the collection of approximately 10 ml of peritoneal effluent samples at certain time intervals and a mid-point blood sample. It is labor intensive, requires an extra visit, takes at least four hours to complete, involves invasive blood sampling (e.g., 10 ml of fluid), and uses proxy for all solutes.

The solute transport rates are assessed by the rates of equilibration between the peritoneal capillary blood and dialysate. As a proxy for all solutes, urea, creatinine, glucose, and sometimes sodium, are measured in the collected samples using different analytical tests. Patients are then categorized as high, high-average, low-average, or low transporters based on their solute transport characteristics.

As the PET is very labor-intensive and the time spent in the clinic by the patient to complete the standard PET is long and requires many lab samples, a mini-PET has been developed for follow-ups in response to clinical change. However, this modified version of the PET has shown inconsistencies compared to the standard PET. For both the standard PET and the mini-PET, errors are possible due to sampling, data entry, calculations, and lab measurements. Another drawback is that the lab measurement for certain compounds may be affected by patient conditions that have to be corrected or otherwise managed. For example, creatinine may be incorrect due to high glucose concentrations and a correction factor is required for calculating the true creatinine amount.

The PET is usually performed infrequently because it is very time and resource intensive. The mini-PET, although less labor-intensive, is not able to provide sufficiently accurate results. There is a need for a more convenient, accurate, and less resource-intensive option, that can be performed more frequently, for tracking a patient's peritoneal transport rate over time, and metabolomic analysis of PD effluent offers such an option.

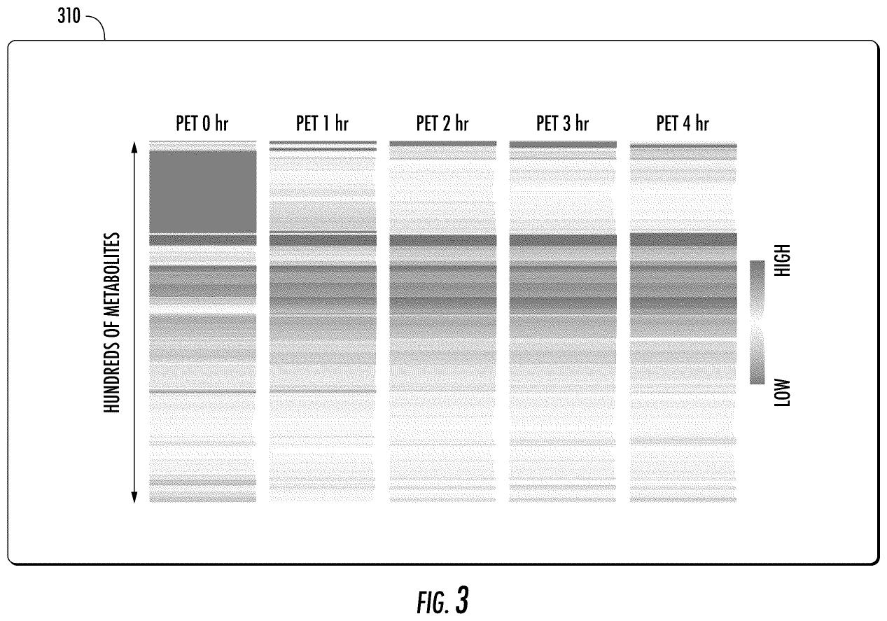

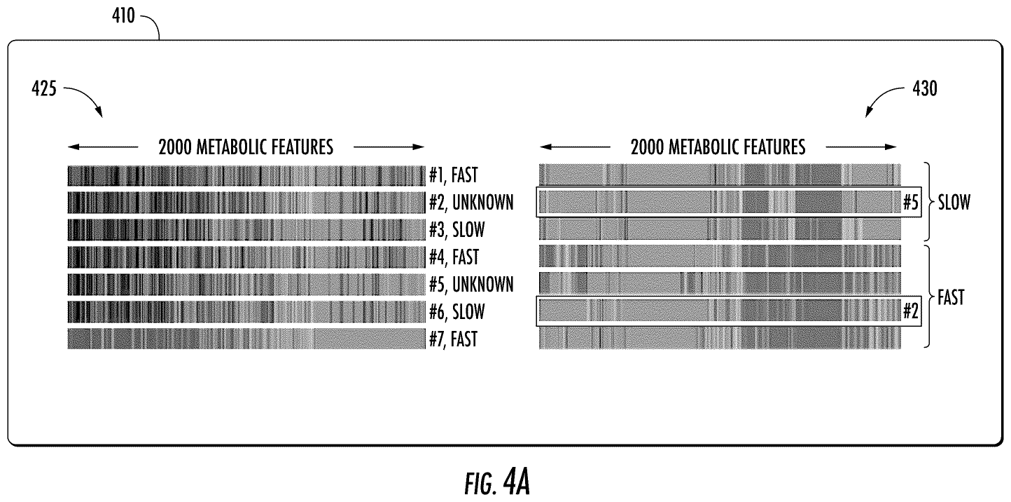

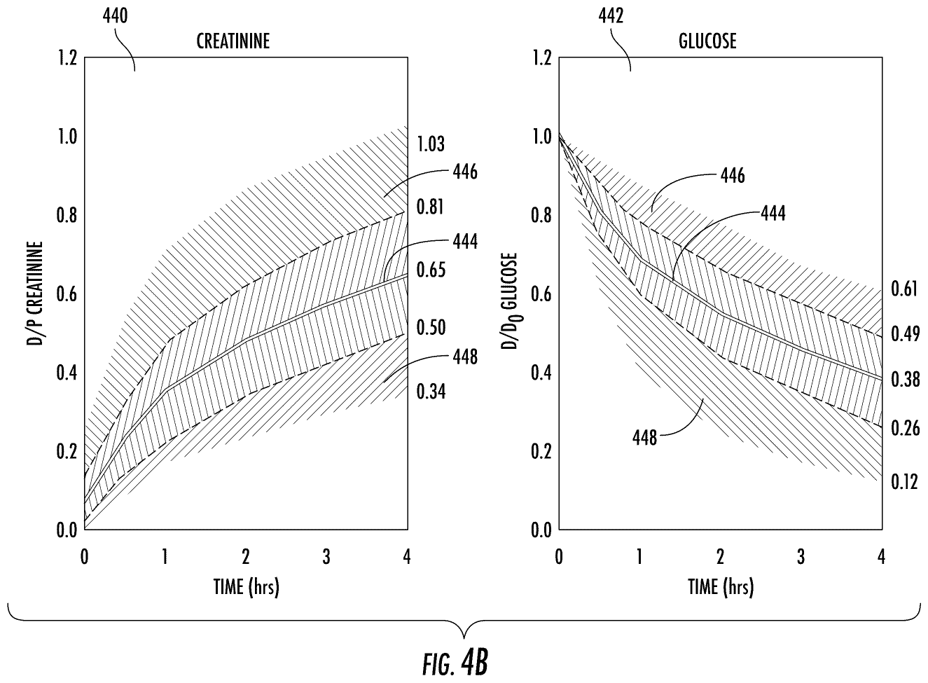

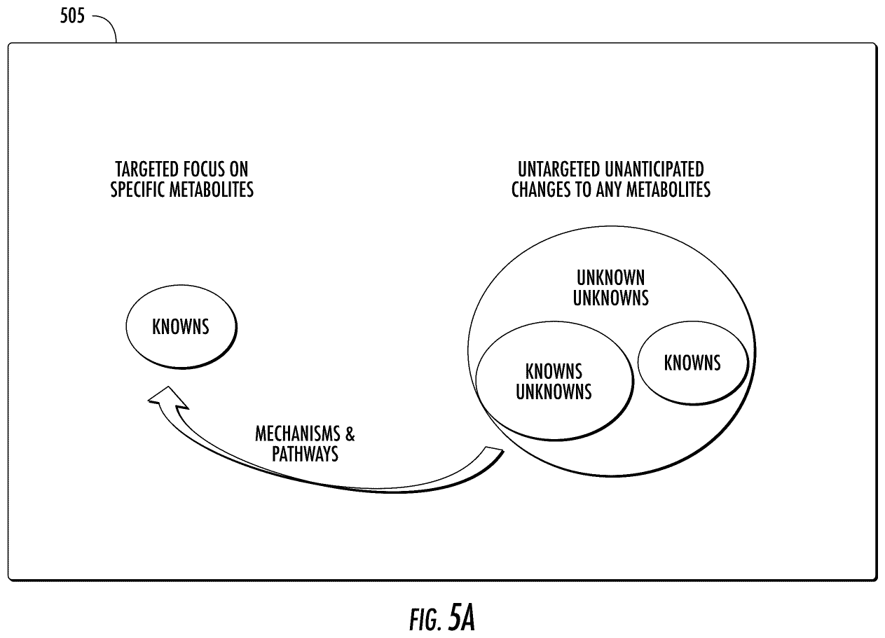

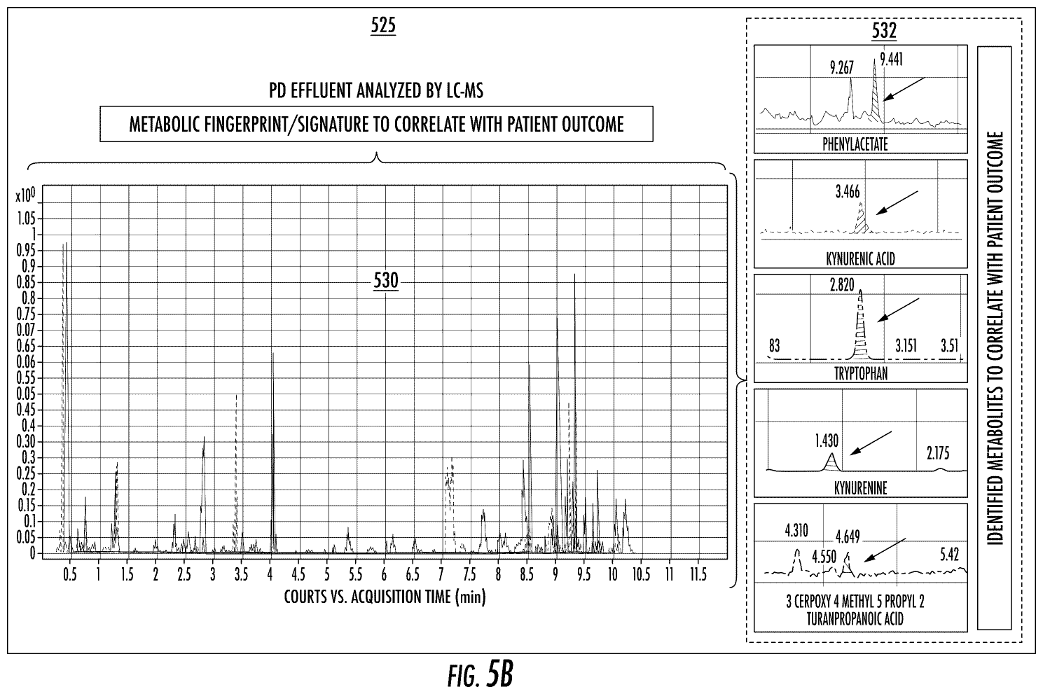

Accordingly, some embodiments may provide a dialysis profile process operative to determine a patient profile that may include a peritoneal transport status in a manner that is more efficient and effective than conventional methods, such as PET. Dialysis profile processes according to some embodiments may provide multiple technological advantages and improvements to technology, including computing technology, over conventional systems. In a non-limiting technological advantage, a dialysis profile process according to some embodiments may provide a more practical and personalized tool to evaluate dialysis adequacy, membrane characteristics, unexplained clinical changes, ultrafiltration failure, and/or the like. In a non-limiting technological advantage, a dialysis profile process according to some embodiments may use PD effluent that is collected from patients while at a clinic for routine checkups and/or the like; accordingly, no extra visits, such as are needed for PET, are required. In addition, the patient and healthcare team do not need to undergo a four-hour protocol. Instead, dialysis profile processes according to some embodiments may use PD effluent that may be routinely collected at scheduled monthly or quarterly visits. In some embodiments, a vast array of molecules (i.e., hundreds of molecules or greater), including, without limitation, urea, creatinine, and glucose, may be analyzed in less than 1 ml of PD effluent using a mass analysis, such as liquid chromatography (LC)-mass spectrometry (MS). In some embodiments, a dialysis profile process may categorize patients, for instance, as high, high-average, average, low-average, or low transporters based on their molecular fingerprints (see, for example, FIGS. 3 and 4). In various embodiments, untargeted and targeted LC-MS, MS, and/or the like approaches may be used to categorize patients (see, for example, FIGS. 5A and 5B). In various embodiments, a dialysis profile process may provide a personalized metabolomics-based transport test for peritoneal dialysis.

Accordingly, dialysis profile processes according to some embodiments may minimize the impact and intrusion of therapy on patients by reducing the number of extra visits to the clinic to determine transport status and providing accurate measurements of physical characteristics important for PD health and effectiveness. In addition, dialysis profile processes according to some embodiments may allow patient transport status to be monitored on a regular and routine basis, instead of only in the presence of warning signs as with conventional methods. As a result, reduced disease maintenance and interventions may lower the risk of infection, which is the second leading cause of death in dialysis patients, and other complications. Accordingly, dialysis profile processes according to some embodiments may operate to improve PD patient quality of life. Other technological advantages are described. Embodiments are not limited in this context.

In addition, dialysis profile processes according to some embodiments may be integrated into multiple practical applications. In one non-limiting practical application, dialysis profile processes may be integrated with providing a personalized metabolomics-based transport test for peritoneal dialysis. In one non-limiting practical application, dialysis profile processes may be integrated with providing a treatment recommendation, dialysis prescription, and/or dialysis treatment may be administered based on the patient characteristic determinations according to some embodiments. In another practical application, a control signal can be sent from an apparatus (e.g., a computing device) to a dialysis machine, PD machine, or dialysis system to provide dialysis treatment on a dialysis patient based on the peritoneal transport status classification of the patient. Other practical applications are described. Embodiments are not limited in this context.

The following Table 1 provides advantages of dialysis profile processes according to some embodiments versus currently available PET tests:

TABLE 1

| | | | Dialysis Profile |
|---|---|---|---|
| | Standard PET | Mini PET | Processes |
| Duration of test | 4 hours | 1 hour | <5 min |
| Sampling time | Multiple | Twice | Once |
| points | | | |
| Specimen | Blood, PD effluent | | PD effluent |
| Volume of effluent | 10 ml | 10 ml | 1 ml (or less) |
| Visits | Extra dialysis access, increased risk of infection | | Routine, no additional |
| | | | connect/disconnect |
| Solutes | Urea, creatine, glucose, | Creatine, glucose | >1000 small |
| | and sometimes sodium | | molecules, including |
| | | | uremic toxins (urea, |
| | | | creatine) and glucose |
| Patient burden | High | Low | None |
| Staff burden | High | Low | None |
| Cost | Time commitment, 6 | Time commitment, 3 | Up to 12 lab tests per |
| | lab tests | lab tests | year |
| Frequency | As needed | As needed | Regularly |
| Drawbacks | Labor-intensive; errors | Not equivalent to | N/A |
| | due to sampling, data | Standard PET | |
| | entry, calculations, and | | |
| | lab measurements; use | | |
| | of proxies to represent | | |
| | all solutes | | |

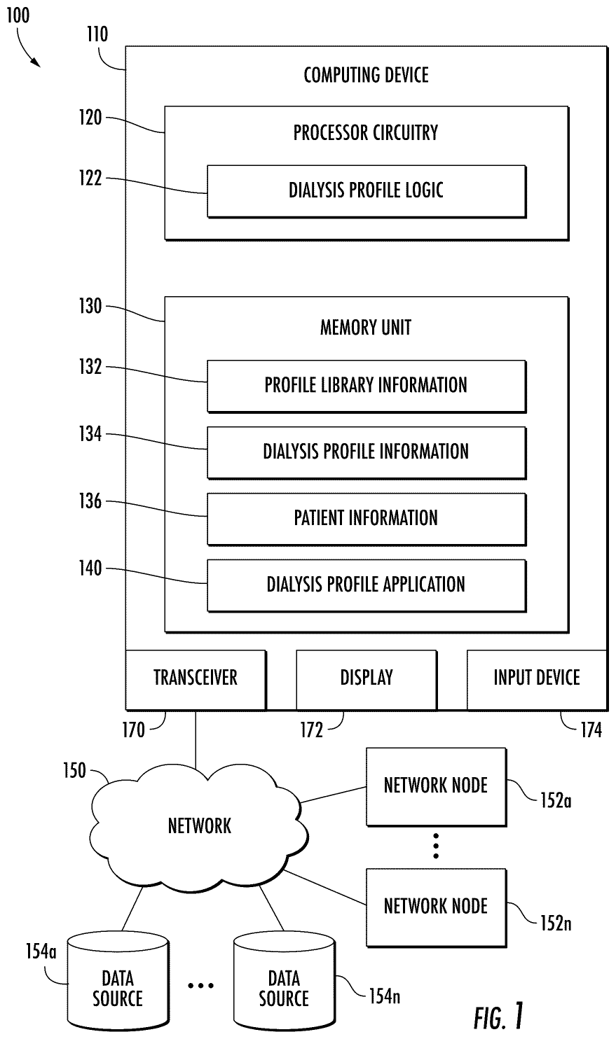

FIG. 1 illustrates an example of an operating environment 100 that may be representative of some embodiments. As shown in FIG. 1, an operating environment may include a computing device 110. In various embodiments, the functions, operations, configurations, data storage functions, applications, logic, and/or the like described with respect to computing device 110 may be performed by and/or stored in one or more other computing devices (not shown), for example, coupled to computing device 110 via a network 150 (i.e., network nodes 152a-n). A single computing device 110 is depicted for illustrative purposes only to simplify the figure. For example, operating environment 100 may include a plurality of computing devices 110 configured independently or in combination to perform aspects of embodiments described herein. Embodiments are not limited in this context.

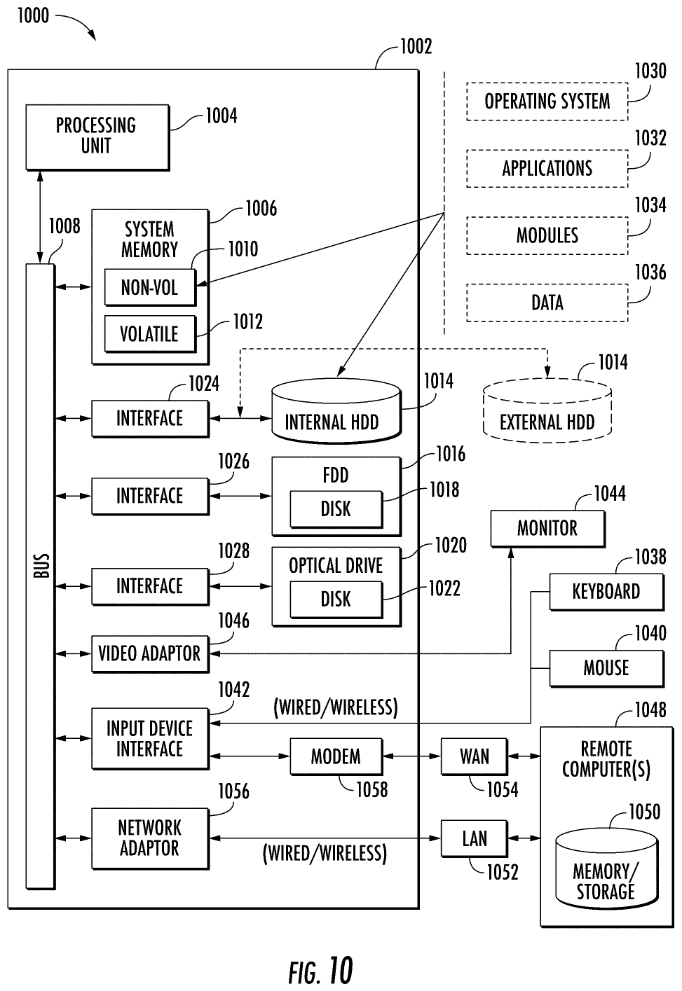

Computing device 110 may include a transceiver 170, a display 172, an input device 174, and/or processor circuitry 120 that may be communicatively coupled to a memory unit 130. Processor circuitry 120 may be, may include, and/or may access various logic for performing processes according to some embodiments. For instance, processor circuitry 120 may include and/or may access a dialysis profile logic 122. Processing circuitry 120 and/or dialysis profile logic 122 and/or portions thereof, may be implemented in hardware, software, or a combination thereof. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution, examples of which are provided by the exemplary computing architecture 1000 in FIG. 10. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, a control loop, a proportional-integral-derivative (PID) controller, combinations of any of the foregoing, and/or the like.

Although dialysis profile logic 122 is depicted in FIG. 1 as being within processor circuitry 120, embodiments are not so limited. For example, dialysis profile logic 122 and/or any component thereof, may be located within an accelerator, a processor core, an interface, an individual processor die, implemented entirely as a software application (for instance, a dialysis profile application 140) and/or the like.

Memory unit 130 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 130 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid state drive (SSD), and/or the like.

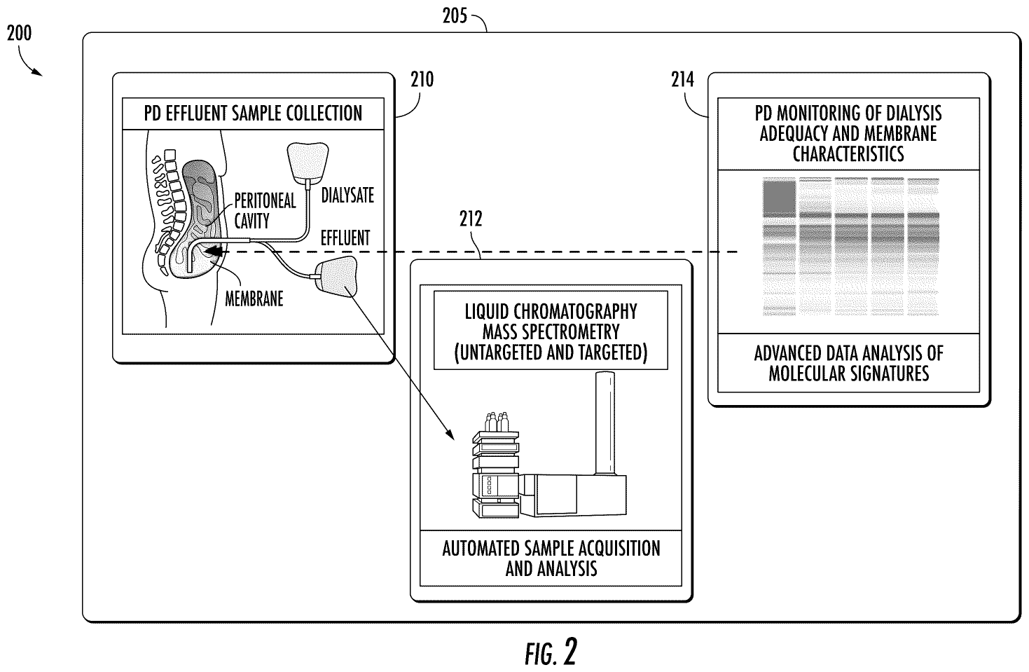

Memory unit 130 may store profile library information 132, patient profile information 134, and/or patient information 136. In some embodiments, profile library information 132 may include information (or “fingerprints”) used as a baseline to determine individual patient profiles (see, for example, FIGS. 2 and 3). In various embodiments, patient profiles may include peritoneal transport status, dialysis adequacy, membrane characteristics, unexplained clinical changes, ultrafiltration failure information and/or classification thereof. For example, a patient profile may include a classification of peritoneal transport status, such as the following categories: high, high-average, average, low-average, or low transporters. Embodiments are not limited to these categories, as patient profiles and/or peritoneal transport status may be categorized using various systems, such as a numeric category, grading (i.e., A-F), symbols, and/or the like. In some embodiments, patient profiles and information associated therewith (i.e., peritoncal transport status) may be stored as patient profile information 132.

In various embodiments, profile library information 132 may include mass analysis information of patients with known patient profiles. For example, profile library information 132 may include MS data of metabolites of patients with a known peritoneal transport status. In various embodiments, the profile library information 132 may include fingerprints, libraries, and/or the like generated from a population of patients so that, for example, patient information may be compared with the same or similar populations of patients (i.e., based on age, gender, disease progression, and/or the like) to determine a patient profile. In some embodiments, the profile library information 132 may include known and unknown biomarkers. Non-limiting examples of known biomarkers may include L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, and 4-hydroxybenzoic acid. In some embodiments, the profile library information 132 may include characteristics of the biomarkers in the population of patients. For example, the biomarkers may include metabolites that demonstrate at least one different characteristic between transport status classifications. A non-limiting characteristic may include at least one of presence/absence (i.e., present in low transporters, but absent in high transporters), concentration (including concentration thresholds), intensity, and regulation (e.g., up-regulation, down-regulation, and/or the like).

In some embodiments, patient information 136 may include information obtained about a patient via analysis of a patient sample, for example, such as blood or PD effluent. For example, in some embodiments, patient information may include MS data resulting from LC-MS and/or MS analysis of a volume of PD effluent. Although LC-MS and MS are used as examples, embodiments are not so limited. In some embodiments, for example, patient information 136 and/or profile library information 132 may be generated via various analytical instrument systems including, without limitation, a liquid chromatography (LC) system, a gas chromatography (GC) system, a mass analyzer system, a mass spectrometer (MS) system, an ion mobility spectrometer (IMS) system, a high-performance liquid chromatography (HPLC) system, a ultra-performance liquid chromatography (UPLC®) system, a ultra-high performance liquid chromatography (UHPLC) system, or any combination thereof.

In some embodiments, the volume of PD effluent required to generate patient information 132 may be about 1 milliliter (ml) or less. In various embodiments, the volume of PD effluent may be about 0.001 ml, about 0.005 ml, about 0.01 ml, about 0.05 ml, about 0.1 ml, about 0.2 ml, about 0.3 ml, about 0.4 ml, about 0.5 ml, about 1.0 ml, about 1.5 ml, and/or any value or range between any two of these values (including endpoints).

In exemplary embodiments, profile library information 132, patient profile information 134, and/or patient information may be obtained from a remote data source, such as data source 154a-n and/or via network node 152a-n.

In some embodiments, dialysis profile logic 122, for example, alone or via dialysis profile application 140 may determine a patient profile for a patient based on patient information 136 and profile library information 132. For example, dialysis profile logic 122 may receive patient information 132 in the form of MS analysis results of a volume of PD effluent from the patient. Dialysis profile logic 122 may compare the MS analysis results to corresponding profile library information 132 (see, for example, FIGS. 2-4) to determine a matching profile. For example, the MS analysis results for Patient A may match with a high peritoneal transport status. Embodiments are not limited in this context.

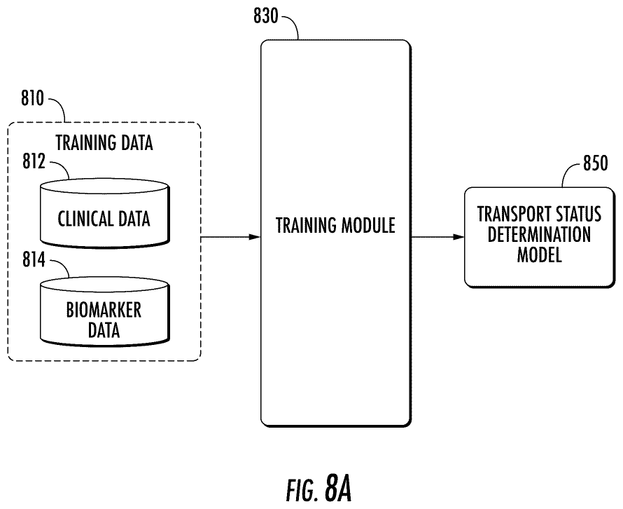

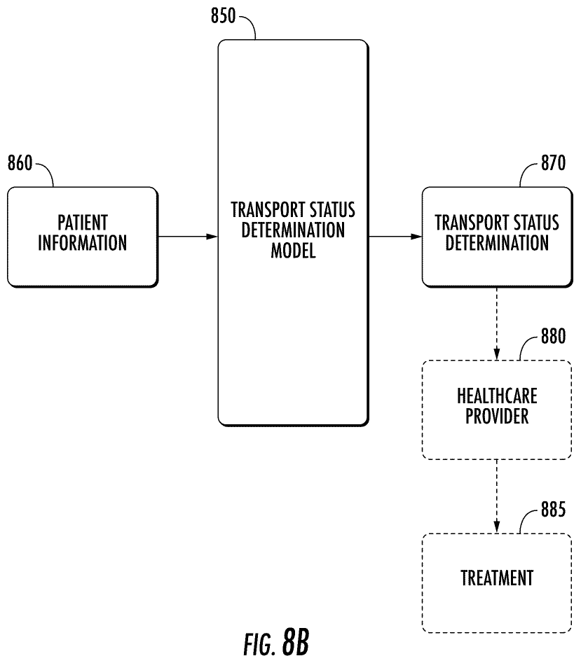

In some embodiments, the memory unit 130 includes instructions (not shown), which when executed by the processor circuitry 120 cause the processor circuitry 120 to perform various operations described herein. For example, in some embodiments, the processor circuitry 120 is configured to access a computational model developed using training data comprising biomarker information of PD patients with known transport statuses to generate output comprising a transport status determination of a patient indicating the PD functionality of the peritoneal membrane of the patient based on input comprising biomarker information of the patient. In some embodiments, the computational model may include the peritoneal transport status determination model 850 of FIG. 8A and FIG. 8B described below. In some embodiments, the computational model may include any other predictive or determination model described herein.

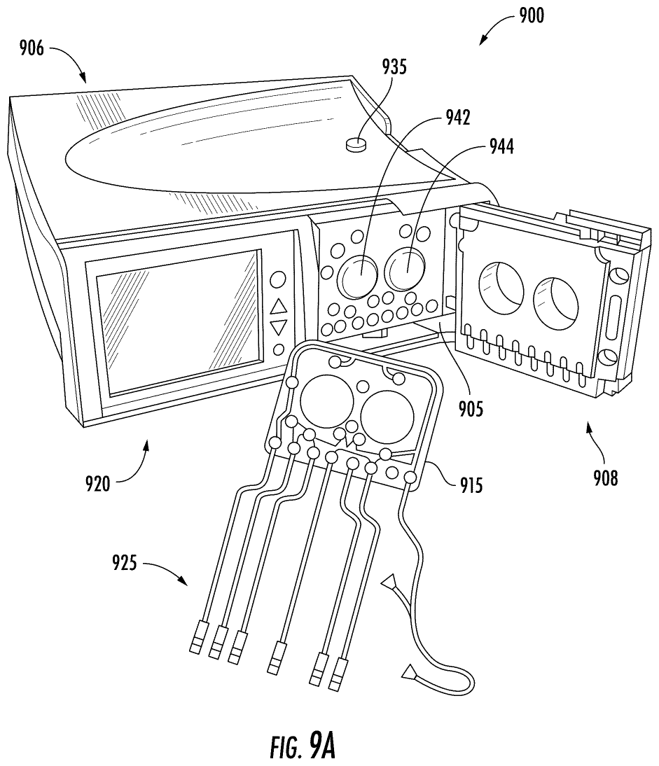

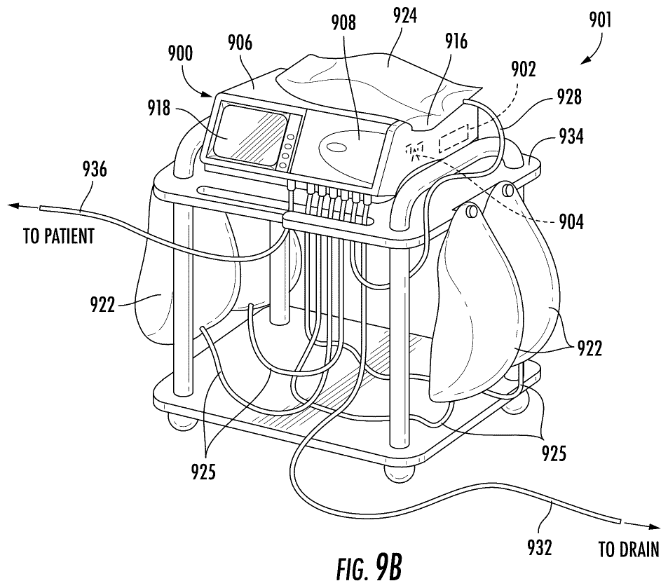

As described below, the computing device 110 can be a standalone device or it can be incorporated into a dialysis machine, such as dialysis machine 900 shown at FIG. 9A. In embodiments, where the computing device 110 is separate from the dialysis machine 900, the dialysis machine 900 and computing device 110 communicate with each other via network 150 or other suitable computer network.

In some embodiments, the processor circuitry 120 is further caused to receive biomarker information of a patient from PD effluent of the patient following a PD treatment. In some embodiments, the training data includes any of the data described above, including information for at least one biomarker, e.g. L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, or 4-hydroxybenzoic acid. In some embodiments, the training data includes at least one characteristic of the at least one biomarker, the at least one characteristic comprising at least one of presence of the at least one biomarker, a concentration of the at least one biomarker, or regulation of the at least one biomarker. In some embodiments, the training data includes data resulting from a peritoneal equilibration test (PET) of PD patients. In some embodiments, the biomarker information includes metabolites that demonstrate at least one different characteristic between peritoneal transport status classifications.

In some embodiments, the computational model includes one or more of a machine learning model; an artificial intelligence model; a neural network; or a convolutional neural network.

In some embodiments, the processor circuitry 120 is configured to determine the peritoneal transport status classification for the patient via providing the biomarker information to the computational model. The computational model then processes the biomarker information and determines the peritoneal transport status classification for the patient. The processor circuitry 120 is then further caused to send a control signal to a dialysis machine to perform a dialysis treatment on the dialysis patient based on the peritoneal transport status classification of the patient profile information.

In some embodiments, the peritoneal transport status classification is selected from a set of classifications indicating a high, high-average, low-average, or low transporter status based on solute transport characteristics. In some other embodiments, the peritoneal transport status classification is indicated using a numerical identifier or symbol.

FIG. 2 illustrates an example of an operating environment 200 that may be representative of some embodiments. As shown in FIG. 2, operating environment 200 depicts a process diagram for a dialysis profile process 205 according to some embodiments.

At block 210, dialysis profile process 205 may include obtaining a patient sample. In some embodiments, the patient sample may include PD effluent. For example, dialysis profile process may use less than 1 ml PD effluent that is collected from patients who come for routine checkups to a clinic (i.e., no extra visit has to be scheduled as compared with conventional methods). At block 212, a vast array of molecules, including urea, creatinine, and glucose, may be analyzed in less than 1 ml of PD effluent using analytical methods such as LC-MS and/or MS. At block 214, analysis of the results (i.e., from block 212) may be performed. For example, dialysis profile process 205 may include monitoring of patient characteristics via advanced data analysis of molecular signatures and/or the like. The advanced data analysis may include evaluating the molecular signatures using machine learning, artificial intelligence, and other techniques described herein to determine the peritoneal transports status classification of the user.

FIG. 3 illustrates PD profiles according to some embodiments. As shown in FIG. 3, PD effluent profiles 310 (for example, a profile library or portion of a profile library) may be generated via analyzing a small amount of PD effluent (0.005 ml) at 0 hr, 1 hr, 2 hr, 3 hr, and 4 hr of a standard PET using LC-MS. Hundreds of molecules may be detected with different abundances. These molecular fingerprints may be used to categorize patient profiles (e.g., peritoneal transport status, dialysis adequacy, membrane characteristics, and/or the like) of PD patients via a dialysis profile process according to some embodiments.

FIG. 4A illustrates PD profiles according to some embodiments. As shown in FIG. 4A, PD profiles 410 may include, for example, a profile library or portion of a profile library. In some embodiments, metabolomics may be used to characterizes PD effluent over time and associate individual temporal changes with a transport status to trigger an adjustment of dialysis prescription or other intervention. For example, dialysis profiling processes according to some embodiments may provide a molecular fingerprinting platform operative to detect metabolites, including, for instance, unknown and/or previously uncategorized metabolites.

In the example of FIG. 4A, seven routinely collected PD effluent samples were analyzed, of which five PD samples had the known transporter type “fast” or “slow” (425). For example, a small amount of PD effluent (0.005 ml) from a routine visit was analyzed using LC-MS. As shown in FIG. 4A, hundreds of molecules were detected with different abundances. Two unknown samples were assigned to either “fast” or “slow” categories based on analysis with known metabolic fingerprints (430). A non-limiting example of an analysis of unknown samples with known metabolic fingerprints may include hierarchical clustering. For example, in 430, hierarchical cluster analysis may delineate differences of molecular fingerprints with peritoneal transport status.

Testing of patient samples, for instance, via PET can determine if a peritoneum has a high, average, or low transport rate. For example, the tests may examine drain volume, how much dextrose is left in the fluid sample, and how much creatinine is in the drain fluid compared with the blood. The following Table 2 provides non-limiting and illustrative peritoneum transport rate characteristics and explains what each level can mean in terms of the type of PD that is best suited for a particular patient:

TABLE 2

| | Waste | Water | |
|---|---|---|---|
| Level | removal | removal | Optimal PD match |
| High | Fast | Poor | APD - Short dwells and frequent |
| | | | exchanges |
| Average | Okay | Okay | APD, CAPD |
| Low | Slow | Good | CAPD |

For example, optimal PD for high transporters involves the use of a PD cycler (e.g., automated PD or APD). Short, fast exchanges will help keep high transporters from absorbing too much PD fluid or dextrose. In another example, for low transporters, optimal PD may be via manual processes, such as CAPD that is spaced out to provide sufficient dwell time to remove wastes. In an additional example, for average transporters, multiple different options (APD or CAPD, for instance) may be selected for treatment.

In addition to or in the alternative to the analysis provided in Table 2, PET results may be analyzed and interpreted using various other processes. In one example, once the PET samples are collected, both the serum and dialysate samples are analyzed for urea, creatinine, and glucose to calculate equilibration ratios. For urea and creatinine, this may be calculated as the dialysate-to-plasma (D-to-P or D/P) ratio wherein the concentration of urea or creatinine in the dialysate at each time point is divided by the concentration of urea or creatinine in the plasma (or serum) sample. For glucose, dividing the dialysate glucose concentration at each time point by the glucose concentration in the 0-hour dialysate sample may provide the equilibration ratios. The equilibration ratios may be used to determine the transport types.

FIG. 4B depicts standardized equilibration curves for creatinine 440 and glucose 442. Line 444 is the average equilibration ratio for that population. One standard deviation up or down (the dashed lines above and below line 444) sets the cutoffs between high and high-average transporters and low and low-average transporters. The outer edges of the first area 446 and second 448 areas represent the maximum and minimums for the group. Generally, patients are categorized based on the 4-hour D/P creatinine and D/D₀ glucose ratios. For example, if the 4-hour D/P creatinine is 0.59 and the 4-hour D/D₀ glucose is 0.47, the patient would be classified as low average. Certain analyses do not use the D/P urea ratios or urea transport classification, but the urea data may be used for prescription modeling. In some examples, the transport type classification is determined to be consistent among the three measures (creatinine, urea, and glucose).

In some examples, the PET information may be used to optimize PD prescriptions. For example, published recommendations of clinical implications for PD prescriptions based on PET transport type are used in conventional methods. High and high-average transporters equilibrate rapidly. Therefore, their transports for urea and creatinine are fast, but it also means they lose the glucose gradient rapidly, so ultrafiltration is limited. These patients would do best with more frequent exchanges with shorter dwells in order to avoid reabsorption. PD Plus (e.g., CAPD and continuous cycling peritoneal dialysis (CCPD)) or Icodextrin should be considered for daytime dwells. It may be possible to have dry periods if the patient has good residual renal function.

On the other hand, low and low-average transporters equilibrate slowly. This means their transports for urea and creatinine are slower, which results in good ultrafiltration with minimal reabsorption, even for a long day dwell. However, such patients may require longer dwells with higher-volume exchanges in order to get adequate clearance.

While the PD prescription has to be individualized for every patient, general recommendations for PD regimens based on PET transport type have been given. For high transporters, short exchanges with a cycler may be well suited and it may be possible to have dry periods if there is good residual renal function. High-average transporters may do cycler or manual exchanges, and dry days may be possible in some cases. For low-average, good residual renal clearance with CAPD or CCPD is possible, meaning continuous 24-hour therapy with no dry periods. Without significant residual function, high-dose CAPD, meaning larger-dwell volumes, may be necessary. For low transporters of average body size, high-dose CAPD may work best. For larger low-transport patients, achieving PD adequacy, goals may be more challenging and require some creativity with their individual PD prescription. The aforementioned treatment regimens are for illustrative purposes only. Patients may be treated using different treatment regimens according to some embodiments.

FIGS. 5A and 5B depict approaches for dialysis profiling processes according to some embodiments. Traditionally, hypothesis-driven approaches have been used to categorize transport status targeting known solutes, such as urea, creatinine, and glucose. In an untargeted approach all molecules, including previously unknowns presented in PD effluent, may be used to generate and/or evaluate patient profile information, such as dialysis adequacy, transport characteristics, and/or the like.

FIG. 5A illustrates a diagram 505 depicting some differences between an untargeted approach that focuses on unanticipated changes to any metabolite. This approach provides a mixture of unknown unknowns, known unknowns, and some knowns. FIG. 5A also illustrates a different aspect, whereby a more targeted approach is taken that focuses on specific metabolites. This approach includes various mechanisms and pathways that eliminate the unknown unknowns and known unknowns, and leaves behind just the known variables associated with the specific metabolites.

FIG. 5B includes a series of charts 525 depicting analysis of PD effluent analyzed by LC-MS. The graph 530 depicts a metabolic fingerprint/signature to correlate with a patient outcome. The side graphs 532 depict specific metabolites identified to correlate with the patient outcome. That is, the side graphs 532 call out specific metabolites identified in the graph 530.

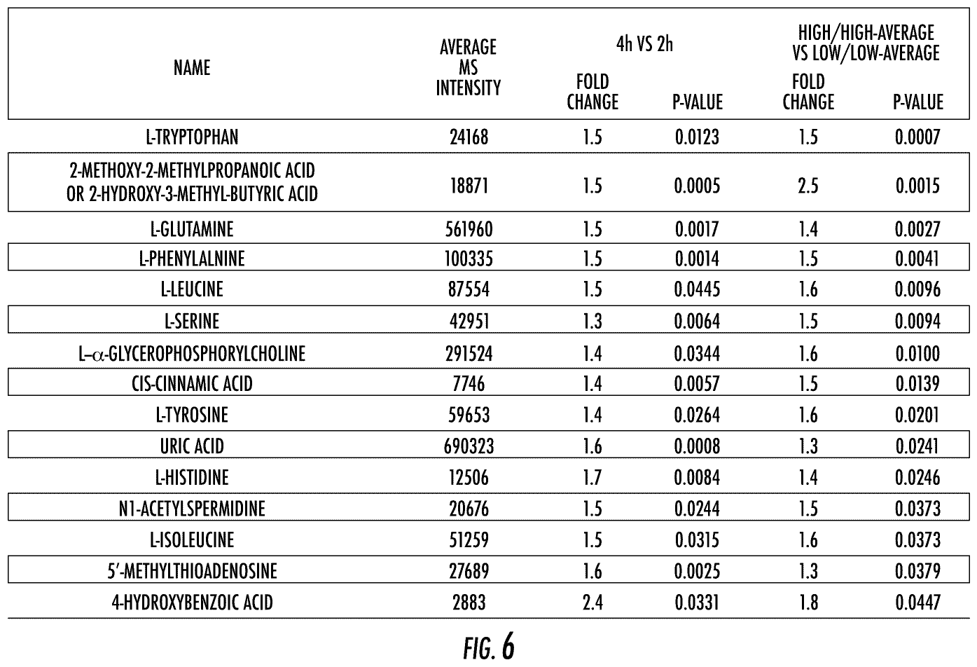

In some embodiments, identified solutes include transport status biomarkers that may be indicative of peritoneal transport status. In various embodiments, the transport status biomarkers include metabolites, for example, found in patient fluids. A non-limiting example of a patient fluid is PD effluent. FIG. 6 depicts non-limiting example transport status biomarkers, including L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, and 4-hydroxybenzoic acid.

The transport status biomarkers may include metabolites or other compounds that demonstrate at least one different characteristic between transport status classifications. A non-limiting characteristic may include at least one of presence/absence (i.e., present in low transporters, but absent in high transporters), concentration (including concentration thresholds), intensity, and regulation (e.g., up-regulation, down-regulation, and/or the like).

The transport status biomarkers may be determined as biomarkers (e.g., established as being indicative of different transport status) or evaluated (e.g., against patient biomarker information to make a prediction or treatment plan for the patient) based on one or more biomarker factors. In one example, transport status biomarkers may be determined or evaluated based on their presence/absence in the fluid samples of patients with different transport statuses. For instance, Biomarker A may be present in patients (e.g., in patient PDE samples) with a low transport status and may be absent in patients with a high transport status. In one example, the transport status biomarkers may be determined or evaluated as biomarkers due to their concentration in patients with different transport statuses. For instance, Biomarker B may have a concentration above a threshold concentration in patients with a low transport status and may have a concentration below the threshold concentration in patients with a high transport status. In one example, the transport status biomarkers may be determined as biomarkers due to differential regulation in patients with different transport statuses. For instance, Biomarker C may be up-regulated (e.g., an increase in concentration after a certain number or duration of PD treatments) in patients with a low transport status and may not be up-regulated (or may be down-regulated) in patients with a high transport status. The aforementioned biomarker factors are non-limiting examples. Other biomarker factors, and associated properties (e.g., timing of regulation, thresholds, and/or the like), are contemplated in the present disclosure.

A transport status library, heatmap, fingerprint, data clusters, AI/ML model training data and/or any other information used to evaluate or determine transport status may be based on the one or more various biomarker factors (and/or associated biomarker factor properties). For example, the concentration of Biomarker D may have predictive or determinative value for transport status. Accordingly, the regulation of Biomarker D of a patient may be compared to a transport status library that includes Biomarker D regulation information of a population of patients with known transport statuses to determine the transport status of the patient.

### Case Study: Transport Status Biomarkers

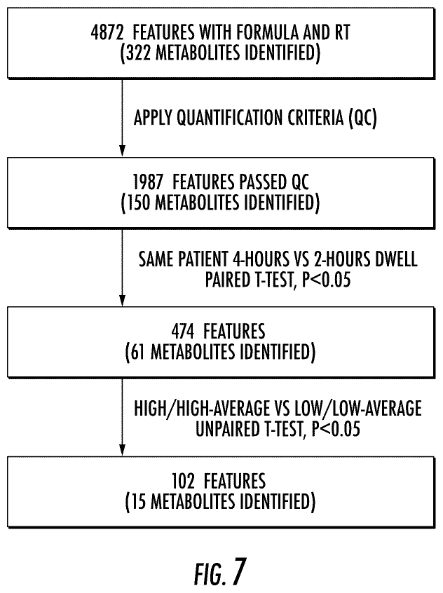

Sixteen PD patients participated in the Case Study. FIG. 7 depicts a workflow for identifying metabolite biomarkers using data determined via the Case Study.

In this case study, all patients completed a 2-hour PD session using 2-liter 2.5% dextrose dialysis fluid. Additionally, six patients underwent a separate 4-hour PD session with the same solution. PD effluent (PDE) was collected at the end of each PD session, with serum collected midway. Patients' transport types were determined using dialysate to serum of creatinine and D/D₀ of glucose at 2 hours and classified as high/high-average (H/HA) and low/low-average (L/LA) group. Untargeted metabolomics of PDE was conducted using quantitative LC-MS.

A total of 1,987 features (predicted formula and retention time) met quantification criteria. Features with intensities differing between the 2-hour and 4-hour PD sessions within the same patient were selected by employing a paired t-test (p<0.05) for 6 patients. Subsequently, the remaining 474 significantly changed features were compared between the H/HA and L/LA groups using an unpaired t-test (see, for example, FIG. 7). As a result, significant differences (p<0.05) in 102 features between the two transport types were observed. Among these, 15 metabolites were identified (see, FIG. 6).

Analysis revealed 150 metabolites in PD effluent. The 15 metabolites depicted in FIG. 6 represent illustrative and non-limiting biomarkers for indicating transport status and/or membrane function under the same prescription regimen.

In some embodiments, dialysis profile processes may be combined with machine learning (ML) techniques, including, without limitation, artificial intelligence (AI) processes, neural networks (NN), and/or the like. For example, dialysis profile processes, patient information, profile information, library information, fingerprints, and/or the like may be used in ML/AI applications to analyze, predict, or otherwise patient profiles (e.g., peritoneal transport status and/or classification thereof) and/or to determine a recommended treatment or other course of action based on a patient profile. In various embodiments, library information may be or may include patient profile computational models (e.g., one or more ML models, one or more AI models, one or more neural networks (NNs), one or more convoluted neural networks (CNNs), and/or the like. In some embodiments, for example. ML/AI processes may correlate the specific molecular patterns with peritoncal transport status.

For example, in some embodiments, ML/AI algorithms, processes, and/or the like may be used to learn the optimal parameters of the predictive model by investigating past examples with known inputs and known outputs. After training, the predictive model can be used to make predictions on unseen inputs (i.e., generalization). For example, dialysis profile processes may involve a classification supervised learning problem in which the output belongs to a set of distinct classes (e.g., transporter type of a PD patient). Non-limiting types of ML algorithms for building predictive models according to some embodiments may include, without limitation, logistic regression, tree-based methods, Random Forest methods, Gradient Boosting methods, deep learning (DL) algorithms such as Recurrent Neural Networks (RNNs), which process sequence of input, and/or the like. Embodiments are not limited in this context.

FIG. 8A illustrates an example of a processing flow in accordance with embodiments described in the present disclosure. More specifically, FIG. 8A illustrates an example of a processing flow for training a peritoneal transport status determination model 850. As shown in FIG. 8A, a training module 830 may be configured to access training data 810. In various embodiments, the training data 810 includes real-world clinical data 812 associated with patients receiving PD. For example, the training data 810 may include PET results of PD patients to determine biomarker information for patients with known transport statuses (i.e., as determine by the PET tests). In some embodiments, clinical data 812 includes known transport statuses, including of patients with different characteristics. Non-limiting examples of characteristics include PD dialysis solution ingredients, concentrations, etc., PD prescription, length of time patient has been receiving PD treatment, demographic information, medical information, comorbidities, and/or the like. In some embodiments, the clinical data 812 includes patient information, including demographics, age, gender, health condition(s), physical characteristics, medications, dialysis treatment, and/or the like.

In some embodiments, the training data 810 includes biomarker data (which may be or may include transport status biomarker information) 814 of patients, for example, associated with the clinical data 812. The biomarker data 814 include targeted metabolites and other compounds, untargeted metabolites and other compounds, concentrations thereof, intensities thereof, regulation information thereof, and/or the like.

The training data 810 may include biomarkers (via the biomarker data 814) associated with known transport statuses (via the clinical data 812).

The training data 810 is fed into the training module 830 configured to train the peritoneal transport status determination model 850. The peritoneal transport status determination model 850 may include various types of data models, mathematical models, regression models, algorithms, AI models, ML models, and/or the like. The peritoneal transport status determination model 850 may be trained to determine a patient transport status (e.g., low, high, average, combinations thereof, numerical value, or symbol, etc.) based on patient biomarkers (e.g., MS profile of patient fluid, such as PDE; distribution of biomarkers; biomarker regulation, and/or the like) alone or in combination with patient information (e.g., demographics, age, gender, health condition(s), PD prescription, and/or the like).

FIG. 8B illustrates an example of a processing flow in accordance with embodiments described in the present disclosure. More specifically, FIG. 8B illustrates an example of a processing flow a transport status determination. As shown in FIG. 8B, patient information 860 may be fed into a trained peritoneal transport status determination model 850. The patient information 860 may include patient information (e.g., demographics, age, gender, health condition(s), and/or the like) and patient biomarkers (e.g., MS analysis of patient fluid, biomarker regulation, and/or the like). The peritoneal transport status determination model 850 may operate to generate a peritoneal transport status determination 870 configured to indicate a peritoneal transport status of the patient. In some embodiments, the peritoneal transport status determination 870 may indicate a probability or confidence in the determination.

In various embodiments, the peritoneal transport status determination 870 may be configured to provide treatment recommendations for the patient for optimal PD treatment. For example, the peritoneal transport status determination 870 may recommend a certain PD treatment for the patient (e.g., APD or CAPD) and particular dialysis solution ingredients and concentrations thereof.

In some embodiments, a healthcare provider 880 may access the peritoneal transport status determination 870 for use in treating a patient 885. For example, a doctor may recommend a particular type of PD treatment, PD prescription, PD dialysis solution, and/or the like. A healthcare professional may treat a patient according to the treatment recommendation to provide an optimal PD treatment specifically configured for the patient biomarker fingerprint.

FIGS. 9A and 9B show an example of a PD system 901, which is configured in accordance with an exemplary embodiment of the system described herein. In some implementations, the PD system 901 may be a home PD system, e.g., a PD system configured for use at a patient's home. The dialysis system 901 may include a dialysis machine 900 (e.g., a peritoneal dialysis machine 900, also referred to as a PD cycler) and in some embodiments the machine may be seated on a cart 934.

The dialysis machine 900 may include a housing 906, a door 908, and a cartridge interface including pump heads 942, 944 for contacting a disposable cassette, or cartridge 915, where the cartridge 915 is located within a compartment formed between the cartridge interface and the closed door 908 (e.g., cavity 905). Fluid lines 925 may be coupled to the cartridge 915 in a known manner, such as via a connector, and may further include valves for controlling fluid flow to and from fluid bags including fresh dialysate and warming fluid. In another embodiment, at least a portion of the fluid lines 925 may be integral to the cartridge 915. Prior to operation, a user may open the door 908 to insert a fresh cartridge 915, and to remove the used cartridge 915 after operation.

The cartridge 915 may be placed in the cavity 905 of the machine 900 for operation. During operation, dialysate fluid may be flowed into a patient's abdomen via the cartridge 915, and spent dialysate, waste, and/or excess fluid may be removed from the patient's abdomen via the cartridge 915. The door 908 may be securely closed to the machine 900. Peritoneal dialysis for a patient may include a total treatment of approximately 10 to 30 liters of fluid, where approximately 2 liters of dialysate fluid are pumped into a patient's abdomen, held for a period of time, e.g., about an hour, and then pumped out of the patient. This is repeated until the full treatment volume is achieved, and usually occurs overnight while a patient sleeps.

A heater tray 916 may be positioned on top of the housing 906. The heater tray 916 may be any size and shape to accommodate a bag of dialysate (e.g., a 5 L bag of dialysate) for batch heating. The dialysis machine 900 may also include a user interface such as a touch screen 918 and control panel 920 operable by a user (e.g., a caregiver or a patient) to allow, for example, set up, initiation, and/or termination of a dialysis treatment. In some embodiments, the heater tray 916 may include a heating element 935, for heating the dialysate prior to delivery into the patient.

Dialysate bags 922 may be suspended from hooks on the sides of the cart 934, and a heater bag 924 may be positioned in the heater tray 916. Hanging the dialysate bags 922 may improve air management as air content may be disposed by gravity to a top portion of the dialysate bag 922. Although four dialysate bags 922 are illustrated in FIG. 9B, any number “n” of dialysate bags may be connectable to the dialysis machine 900 (e.g., 1 to 5 bags, or more), and reference made to first and second bags is not limiting to the total number of bags used in a dialysis system 901. For example, the dialysis machine may have dialysate bags 922a, . . . 922n connectable in the system 901. In some embodiments, connectors and tubing ports may connect the dialysate bags 922 and lines for transferring dialysate. Dialysate from the dialysate bags 922 may be transferred to the heater bag 924 in batches. For example, a batch of dialysate may be transferred from the dialysate bags 922 to the heater bag 924, where the dialysate is heated by the heating element 935. When the batch of dialysate has reached a predetermined temperature (e.g., approximately 98°-100° F., 37° C.), the batch of dialysate may be flowed into the patient. The dialysate bags 922 and the heater bag 924 may be connected to the cartridge 915 via dialysate bag lines or tubing 925 and a heater bag line or tubing 928, respectively. The dialysate bag lines 925 may be used to pass dialysate from dialysate bags 922 to the cartridge during use, and the heater bag line 928 may be used to pass dialysate back and forth between the cartridge and the heater bag 924 during use. In addition, a patient line 936 and a drain line 932 may be connected to the cartridge 915. The patient line 936 may be connected to a patient's abdomen via a catheter and may be used to pass dialysate back and forth between the cartridge and the patient's peritoneal cavity by the pump heads 942, 944 during use. The drain line 932 may be connected to a drain or drain receptacle and may be used to pass dialysate from the cartridge to the drain or drain receptacle during use.

Although in some embodiments, dialysate may be batch heated as described above, in other embodiments, dialysis machines may heat dialysate by in-line heating, e.g., continuously flowing dialysate through a warmer pouch positioned between heating elements prior to delivery into a patient. For example, instead of a heater bag for batch heating being positioned on a heater tray, one or more heating elements may be disposed internal to the dialysis machine. A warmer pouch may be insertable into the dialysis machine via an opening. It is also understood that the warmer pouch may be connectable to the dialysis machine via tubing (e.g., tubing 925), or fluid lines, via a cartridge. The tubing may be connectable so that dialysate may flow from the dialysate bags, through the warmer pouch for heating, and to the patient.

In such in-line heating embodiments, a warmer pouch may be configured so dialysate may continually flow through the warmer pouch (instead of transferred in batches for batch heating) to achieve a predetermined temperature before flowing into the patient. For example, in some embodiments the dialysate may continually flow through the warmer pouch at a rate between approximately 100-300 mL/min. Internal heating elements (not shown) may be positioned above and/or below the opening, so that when the warmer pouch is inserted into the opening, the one or more heating elements may affect the temperature of dialysate flowing through the warmer pouch. In some embodiments, the internal warmer pouch may instead be a portion of tubing in the system that is passed by, around, or otherwise configured with respect to, a heating element(s).

The touch screen 918 and the control panel 920 may allow an operator to input various treatment parameters to the dialysis machine 900 and to otherwise control the dialysis machine 900. In addition, the touch screen 918 may serve as a display. The touch screen 918 may function to provide information to the patient and the operator of the dialysis system 901. For example, the touch screen 918 may display information related to a dialysis treatment to be applied to the patient, including information related to a prescription.

The dialysis machine 900 may include a processing circuit 902 that resides inside the dialysis machine 900, the processing circuit 902 being configured to communicate with the touch screen 918 and the control panel 920. The processing circuit 902 may be configured to receive data from the touch screen 918 the control panel 920 and sensors, e.g., weight, air, flow, temperature, and/or pressure sensors, and control the dialysis machine 900 based on the received data. For example, the processing circuit 902 may adjust the operating parameters of the dialysis machine 900. In some embodiments, the computing device 110 of FIG. 1 can be integrated into the dialysis machine 900 and the processing circuit 902 of the dialysis machine 900 can perform the same functions as the processor circuitry 120 of FIG. 1. For example, the dialysis machine 900 can process the control signal sent by the computing device 110 and effectuate the dialysis treatment defined by the control signal based on the peritoneal transport status classification of the dialysis patient.

The dialysis machine 900 may be configured to connect to a network 903. The connection to network 903 may be via a wired and/or wireless connection. The dialysis machine 900 may include a connection component 904 configured to facilitate the connection to the network 903. The connection component 904 may be a transceiver for wireless connections and/or other signal processor for processing signals transmitted and received over a wired connection. Other medical devices (e.g., other dialysis machines) or components may be configured to connect to the network 903 and communicate with the dialysis machine 900. The user interface portion such as the touch screen 918 and/or control panel 920 may include one or more buttons for selecting and/or entering user information. The touch screen 918 and/or control panel 920 may be operatively connected to a controller (not shown) and disposed in the machine 900 for receiving and processing the inputs to operate the dialysis machine 900.

In some embodiments, the dialysis machine 900 can receive the control signal from the computing device 110 described above in FIG. 1 over the network 903. The processing circuit 902 of the dialysis machine 900 will then process the control signal and control operations of the dialysis machine 900 to effectuate the dialysis treatment defined by the control signal based on the peritoneal transport status classification of the dialysis patient.

FIG. 10 illustrates an embodiment of an exemplary computing architecture 1000 suitable for implementing various embodiments as previously described. In various embodiments, the computing architecture 1000 may comprise or be implemented as part of an electronic device. In some embodiments, the computing architecture 1000 may be representative, for example, of computing device 1002 and/or components thereof. The embodiments are not limited in this context.

As used in this application, the terms “system” and “component” and “module” are intended to refer to a computer-related entity, either hardware, a combination of hardware and software, software, or software in execution, examples of which are provided by the exemplary computing architecture 1000. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 1000 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 1000.

As shown in FIG. 10, the computing architecture 1000 comprises a processing unit 1004, a system memory 1006 and a system bus 1008. The processing unit 1004 can be any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Intel® Celeron®, Core (2) Duo®, Itanium®, Pentium®, Xeon®, and XScalc® processors; and similar processors. Dual microprocessors, multi-core processors, and other multi-processor architectures may also be employed as the processing unit 1004.

The system bus 1008 provides an interface for system components including, but not limited to, the system memory 1006 to the processing unit 1004. The system bus 1008 can be any of several types of bus structure that may further interconnect to a memory bus (with or without a memory controller), a peripheral bus, and a local bus using any of a variety of commercially available bus architectures. Interface adapters may connect to the system bus 1008 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 1006 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 10, the system memory 1006 can include non-volatile memory 1010 and/or volatile memory 1012. A basic input/output system (BIOS) can be stored in the non-volatile memory 1010.

The computer 1002 may include various types of computer-readable storage media in the form of one or more lower speed memory units, including an internal (or external) hard disk drive (HDD) 1014, a magnetic floppy disk drive (FDD) 1016 to read from or write to a removable magnetic disk 1018, and an optical disk drive 1020 to read from or write to a removable optical disk 1022 (e.g., a CD-ROM or DVD). The HDD 1014, FDD 1016 and optical disk drive 1020 can be connected to the system bus 1008 by an HDD interface 1024, an FDD interface 1026 and an optical drive interface 1029, respectively. The HDD interface 1024 for external drive implementations can include at least one or both of Universal Serial Bus (USB) and IEEE 1384 interface technologies.

The drives and associated computer-readable media provide volatile and/or nonvolatile storage of data, data structures, computer-executable instructions, and so forth. For example, a number of program modules can be stored in the drives and memory units 1010, 1012, including an operating system 1030, one or more application programs 1032, other program modules 1034, and program data 1036. In one embodiment, the one or more application programs 1032, other program modules 1034, and program data 1036 can include, for example, the various applications and/or components of computing device 110.

A user can enter commands and information into the computer 1002 through one or more wire/wireless input devices, for example, a keyboard 1038 and a pointing device, such as a mouse 1040. Other input devices may include microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, trackpads, sensors, styluses, and the like. These and other input devices are often connected to the processing unit 1004 through an input device interface 1042 that is coupled to the system bus 1008, but can be connected by other interfaces such as a parallel port, IEEE 994 serial port, a game port, a USB port, an IR interface, and so forth.

A monitor 1044 or other type of display device is also connected to the system bus 1008 via an interface, such as a video adaptor 1046. The monitor 1044 may be internal or external to the computer 1002. In addition to the monitor 1044, a computer typically includes other peripheral output devices, such as speakers, printers, and so forth.

The computer 1002 may operate in a networked environment using logical connections via wire and/or wireless communications to one or more remote computers, such as a remote computer 1049. The remote computer 1049 can be a workstation, a server computer, a router, a personal computer, portable computer, microprocessor-based entertainment appliance, a peer device or other common network node, and typically includes many or all of the elements described relative to the computer 1002, although, for purposes of brevity, only a memory/storage device 1050 is illustrated. The logical connections depicted include wire/wireless connectivity to a local area network (LAN) 1052 and/or larger networks, for example, a wide area network (WAN) 1054. Such LAN and WAN networking environments are commonplace in offices and companies, and facilitate enterprise-wide computer networks, such as intranets, all of which may connect to a global communications network, for example, the Internet.

When used in a LAN networking environment, the computer 1002 is connected to the LAN 1052 through a wire and/or wireless communication network interface or adaptor 1056. The adaptor 1056 can facilitate wire and/or wireless communications to the LAN 1052, which may also include a wireless access point disposed thereon for communicating with the wireless functionality of the adaptor 1056.

When used in a WAN networking environment, the computer 1002 can include a modem 1058, or is connected to a communications server on the WAN 1054, or has other means for establishing communications over the WAN 1054, such as by way of the Internet. The modem 1059, which can be internal or external and a wire and/or wireless device, connects to the system bus 1008 via the input device interface 1042. In a networked environment, program modules depicted relative to the computer 1002, or portions thereof, can be stored in the remote memory/storage device 1050. It will be appreciated that the network connections shown are exemplary and other means of establishing a communications link between the computers can be used.

The computer 1002 is operable to communicate with wire and wireless devices or entities using the IEEE 802 family of standards, such as wireless devices operatively disposed in wireless communication (e.g., IEEE 802.16 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

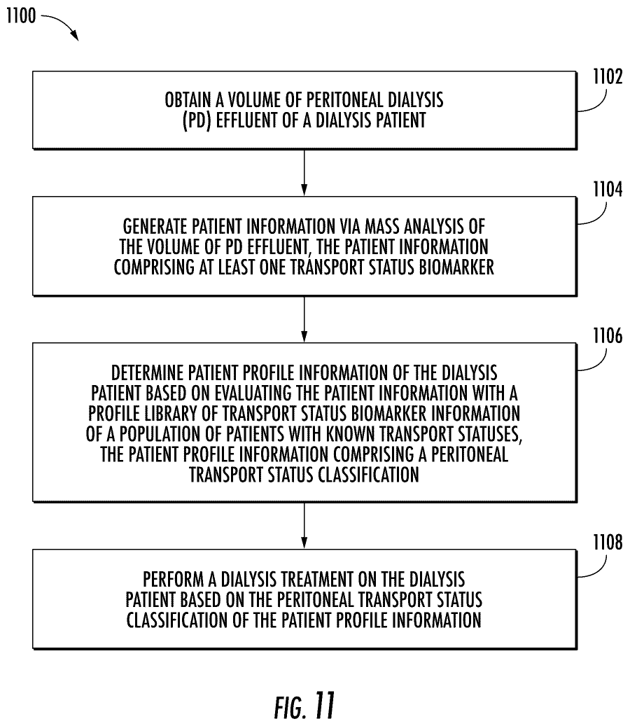

FIG. 11 is a flow chart illustrating some processes executed in an example method 1100 according to some embodiments of the present disclosure. For example, as shown at block 1102, the method 1100 includes obtaining a volume of PD effluent of a dialysis patient. As shown at block 1104, the method 1100 includes generating patient information via mass analysis of the volume of PD effluent, the patient information comprising at least one transport status biomarker. As shown at block 1106, the method 1100 includes determining patient profile information of the dialysis patient based on evaluating the patient information with a profile library of transport status biomarker information of a population of patients with known transport statuses, the patient profile information comprising a peritoneal transport status classification. And as shown at block 1108, the method 1100 includes performing a dialysis treatment on the dialysis patient based on the peritoneal transport status classification of the patient profile information.

In some embodiments, the transport status biomarker information comprises information for at least one biomarker, the at least one biomarker including at least one of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N-acetylspermidine, L-isoleucine, 5′-methylthioadenosine, and 4-hydroxybenzoic acid.

In some embodiments, the transport status biomarker information comprises at least one characteristic of the at least one biomarker, the at least one characteristic comprising at least one of presence of the at least one biomarker, a concentration of the at least one biomarker, or regulation of the at least one biomarker.

In some embodiments, the peritoneal transport status classification is selected from a set of classifications indicating a high, high-average, low-average, or low transporter status based on solute transport characteristics; or the peritoneal transport status classification is indicated using a numerical identifier or symbol. In some embodiments, the method 1100 further comprises determining a dialysis prescription based on the peritoneal transport status classification. In some embodiments, the method 1100 further comprises determining a type of PD based on the peritoneal transport status classification; and performing the dialysis treatment on the dialysis patient according to the determined type of PD. In some embodiments, the method 1100 further comprises determining a PD solution based on the peritoneal transport status classification; and performing the dialysis treatment on the dialysis patient according to the determined PD solution.

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A method comprising:
obtaining a volume of peritoneal dialysis (PD) effluent of a dialysis patient;
generating patient information via mass analysis of the volume of PD effluent, the patient information comprising at least one transport status biomarker;
determining patient profile information of the dialysis patient based on evaluating the patient information with a profile library of transport status biomarker information of a population of patients with known transport statuses, the patient profile information comprising a peritoneal transport status classification, wherein the transport status biomarker information comprises information for at least one biomarker, the at least one biomarker including at least one of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, or 4-hydroxybenzoic acid; and
performing a dialysis treatment on the dialysis patient based on the peritoneal transport status classification of the patient profile information.

2. The method of claim 1, wherein the peritoneal transport status classification is selected from a set of classifications indicating a high, high-average, low-average, or low transporter status based on solute transport characteristics.

3. The method of claim 1, wherein the transport status biomarker information comprises at least one characteristic of the at least one biomarker, the at least one characteristic comprising at least one of presence of the at least one biomarker, a concentration of the at least one biomarker, or regulation of the at least one biomarker.

4. The method of claim 1, wherein the peritoneal transport status classification is indicated using a numerical identifier or symbol.

5. The method of claim 1, further comprising determining a dialysis prescription based on the peritoneal transport status classification.

6. The method of claim 1, further comprising determining a type of PD based on the peritoneal transport status classification; and
performing the dialysis treatment on the dialysis patient according to the determined type of PD.

7. The method of claim 1, further comprising determining a PD solution based on the peritoneal transport status classification; and
performing the dialysis treatment on the dialysis patient according to the determined PD solution.

8. An apparatus, comprising:
a processing circuit; and
a memory coupled to the processing circuit, the memory comprising instructions that, when executed by the processing circuit, cause the processing circuit to:
access a computational model trained using training data comprising biomarker information of peritoneal dialysis (PD) patients with known transport statuses to generate output comprising a transport status determination of a patient indicating the PD functionality of the peritoneal membrane of the patient based on input comprising biomarker information of the patient, wherein the biomarker information includes information on at least one of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, or 4-hydroxybenzoic acid;
receive biomarker information of a patient from PD effluent of the patient following a PD treatment;
determine the peritoneal transport status classification for the patient via providing the biomarker information to the computational model; and
send a control signal to a dialysis machine to perform a dialysis treatment on the dialysis patient based on the peritoneal transport status classification of the patient profile information.

9. The apparatus of claim 8, wherein the peritoneal transport status classification is selected from a set of classifications indicating a high, high-average, low-average, or low transporter status based on solute transport characteristics.

10. The apparatus of claim 8, the training data comprising at least one characteristic of the at least one biomarker, the at least one characteristic comprising at least one of presence of the at least one biomarker, a concentration of the at least one biomarker, or regulation of the at least one biomarker.

11. The apparatus of claim 8, wherein the computational model includes one or more of:
a machine learning model;
an artificial intelligence model;
a neural network; or
a convolutional neural network.

12. The apparatus of claim 8, wherein the peritoneal transport status classification is indicated using a numerical identifier or symbol.

13. The apparatus of claim 12, wherein the biomarker information includes metabolites that demonstrate at least one different characteristic between peritoneal transport status classifications.

14. The apparatus of claim 8, wherein the training data includes data resulting from a peritoneal equilibration test (PET) of PD patients.

15. A PD machine comprising the apparatus of claim 8, the PD machine configured to process the control signal and effectuate the dialysis treatment based on the peritoneal transport status classification.

16. A non-transitory computer-readable storage medium having executable instructions stored thereon, which when executed by the processing circuit cause the processing circuit to:
access a computational model trained using training data comprising biomarker information of peritoneal dialysis (PD) patients with known transport statuses to generate output comprising a transport status determination of a patient indicating the PD functionality of the peritoneal membrane of the patient based on input comprising biomarker information of the patient, wherein the biomarker information includes information on at least one of L-tryptophan, 2-methoxy-2-methylpopanoic acid, 2-hydroxy-3-methyl-butyric acid, L-glutamine, L-phenylalanine, L-leucine, L-serine, L-α-glycerophosphorylcholine, cis-cinnamic acid, L-tyrosine, uric acid, L-histidine, N1-acetylspermidine, L-isoleucine, 5′-methylthioadenosine, or 4-hydroxybenzoic acid;
receive biomarker information of a patient from PD effluent of the patient following a PD treatment;
determine the peritoneal transport status classification for the patient via providing the biomarker information to the computational model; and
send a control signal to a dialysis machine to perform a dialysis treatment on the dialysis patient based on the peritoneal transport status classification of the patient profile information.

17. The non-transitory computer-readable storage medium of claim 16, wherein the peritoneal transport status classification is selected from a set of classifications indicating a high, high-average, low-average, or low transporter status based on solute transport characteristics.

18. The non-transitory computer-readable storage medium of claim 16, wherein the training data comprises at least one characteristic of the at least one biomarker, the at least one characteristic comprising at least one of presence of the at least one biomarker, a concentration of the at least one biomarker, or regulation of the at least one biomarker.

19. The non-transitory computer-readable storage medium of claim 16, wherein the computational model includes one or more of:
a machine learning model;
an artificial intelligence model;
a neural network; or
a convolutional neural network.

20. The non-transitory computer-readable storage medium of claim 16, wherein the peritoneal transport status classification is indicated using a numerical identifier or symbol.
